(19) United States
(12) Patent Application Publication
(10) Pub. No.: US 2003/0139716 A1
(43) Pub. Date: Jul. 24, 2003

(54) Absorbent product

(76) Inventor: Torgny Falk, Hisings-Backa (SE)

Correspondence Address: BURNS, DOANE, SWECKER & MATHIS, L.L.P., P.O. BOX 1404, Alexandria, VA, 22313-1404, US

(21) Appl. No.: 10/304,798

(22) Filed: Nov. 27, 2002

Related U.S. Application Data
(60) Provisional application No. 60/333,129, filed on Nov. 27, 2001.

(51) Int. Cl. A61F 13/15

(52) U.S. Cl. 604/368; 604/378

(57) ABSTRACT

An absorbent product, such as a panty liner, a sanitary towel, an incontinence protection, a dressing, or a tampon for absorption of blood or menstrual fluid, includes a liquid permeable surface layer facing the wearer during use, a liquid impermeable backing layer facing away from the wearer during use, and an absorbent body with one or more material layers, positioned between the backing layer and the surface layer. The absorbent body includes super-absorbent polymers (SAP), the SAP material has a contact angle smaller than 60°, as measured with defibrinated sheep blood, and the SAP material has a gel-strength which is higher than 18 kPa. Hereby, a very good absorption of blood-containing liquids is obtained.

### CROSS-REFERENCE TO RELATED APPLICATIONS

The present application claims the priority under 35 U.S.C. §119 of U.S. Provisional Application No. 60/333,129, filed in the United States on Nov. 27, 2001, the entire contents of which are hereby incorporated herein by reference.

### BACKGROUND

1. Technical Field

The invention refers to an absorbent product for absorption of blood or menstrual fluid, intended to be worn in the crotch part of an undergarment, and more particularly for an absorbent product having an absorbent body which includes a super-absorbent polymer.

2. Background Information

In order to absorb fluid in absorbent products, super-absorbents (SAP) are often used. These have a capability to absorb liquid in a quantity several times their own weight, and for this reason they have shown to be very suitable for use in products of this kind. Large amounts of liquid may quickly and efficiently be absorbed. The absorbent core of the products may be relatively small, which makes the product less bulky at use and at package.

Normally, SAP is added to the rest of the core structure in the form of polymer particles. These particles are built up of super-absorbent polymer chains forming a network, which is held together by cross-linkings. Cross-linking agents can be used to obtain these cross-linkings.

For example, DE-A-19941423 discloses a SAP-polymer and its production. The SAP includes (a) 55-99.9% (by weight) of an ethylene-containing polymerizable monomer, (b) 0-40% (by weight) of a monomer co-polymerizable with the ethylene-containing polymerizable monomer, (c) 0.01-5.0%, and preferably 0.1-2.0%, (by weight) of a cross-linking agent, and (d) 0-30% (by weight) of a water-soluble polymer.

Most available absorbent SAP products are adapted for urine absorption. However, embodiments of the invention described herein are primarily intended to absorb blood, which can present different problems compared to urine absorption. In comparison to blood, urine has a much higher water content. Conventional SAPs are adapted for absorption of water.

Blood is a liquid having a high viscosity. Blood includes up to about 45% blood cells, and about 55% of blood plasma. The blood plasma comprises salts, water and proteins (e.g. albumin, IgG and fibrinogen). The proteins add up to about 7-8% of the blood plasma. The amount of protein in blood is on the order of 10¹⁷/ml blood, and the amount of blood cells is on the order of 10⁹/ml blood.

At absorption of blood to a SAP particle, the proteins will interact to a much greater extent with the surface of the SAP particles, compared to the blood cells. SAP particles are primarily adapted for water absorption. Therefore, it is desirable to design the surface of the SAP particles in such a way that the transport of water molecules into the particles is favored. By applying a hydrophilic surface to the SAP particles, the tendency of the proteins to interact with the surface of the particles will decrease. Tests (Nadarajah et al., “Modeling the Dynamics of Proteins Adsorption to Surfaces”, from ACS Symposium Series 602, Proteins at Interfaces II—Fundamentals and Applications (Horbett and Brash, 1995): chapter 13) have shown that a hydrophilic surface results in a lower protein interaction. However, the water molecules are polar, and will therefore interact to a high degree with a hydrophilic surface. The same is applicable for salts, which in water solution occur in ionic form, which also will be attracted by the surface of the SAP particles. In this way, the transport of water and salt to the surface of the SAP particles, and thereby also the possibility for them to be absorbed into the SAP particles, will increase, at the expense of the protein assembly at the surface, if the surface of the SAP particles is hydrophilic. Moreover, a large difference in ion concentration within and outside a SAP particle may result in a high osmotic pressure, which contributes to a strong absorption capacity.

The degree of cross-linking determines the elasticity of the material, as well as its absorbent properties. A higher degree of cross-linking results in a more brittle material, but which also can have a higher initial ability to absorb liquid, i.e., the SAP becomes quick, but has a lower total capacity to absorb a large volume, i.e., a lower total capacity. A low degree of cross-linking can produce a viscous, expandable material, which has the capacity to absorb a large volume of liquid. However, a low degree of cross-linking may result in a relatively slow absorption. A reason for this is that the diffusion constant increases and the swelling is faster (at a higher cross-linking degree), but the total capacity decreases (“Modern superabsorbent polymer technology”, Buchholz and Graham, Wiley-VCH, 1998). A balance between these two extremes is therefore desirable.

A high degree of cross-linking also results in a high gel-strength. The gel-strength is a measure of the ability of the SAP-gel to withstand pressure without losing liquid. A SAP having a high gel-strength thus has a high ability to keep liquid when it is subjected to load, such as when the wearer of an absorbent product sits down or lies in a way that the article, and the SAP particle within it, is pressed together.

U.S. Pat. No. 5,241,009 discloses an absorbent product which may be used for absorption of blood. This document recommends a cross-linking degree of about 0.1-2.0% for blood absorption.

WO94/15651 discloses a SAP being specially adapted for absorption under pressure, which comprises 0.1-5% (by weight) of a cross-linking agent.

U.S. Pat. No. 5,985,432 discloses a SAP having a cross-linking degree of 0.001 to 5 mol %. The absorbent material comprises a polyether or/and a polycation, which is bound to the absorbing polymer, resulting in a contact angle for blood of 0 to 400.

Many of the SAP-containing absorbent products, which are used today for absorption of blood and menstrual liquids, are based on polyacrylic acid, which is a standard SAP material. Other base materials for SAPs exist, such as, for example, CMC (cellulose) or starch.

Thus, today some products for blood absorption exist. However, these are to a great extent also adapted for urine absorption.

### SUMMARY

It is of interest to improve the absorption characteristics for the existing products, not the least by optimization of the chemical construction of the SAP material, in order to provide a SAP material which has improved absorption characteristics for blood.

An object of the invention is to provide an absorbent product, which solves the problems stated above, thereby providing a product having improved absorption properties for blood.

Surprisingly, by using a SAP material having a gel-strength which exceeds 18000 Pa, and having a suitable wettability on the surface of the particles, a strongly improved absorption capacity for blood is obtained, compared to the materials used today.

Accordingly, an embodiment of the invention relates to an absorbent product, such as a panty liner, a sanitary towel, or an incontinence protection for absorption of blood or menstrual fluid, intended to be worn in the crotch part of an undergarment, comprising a liquid permeable surface layer facing the wearer during use, a liquid impermeable backing layer facing away from the wearer during use, and an absorbent body comprising one or more material layers, positioned between the surface layer and the backing layer. The absorbent body can comprise super-absorbent polymers (SAP), with the SAP material having a contact angle at its surface which is smaller than 60°, as measured by fresh, sterile and defibrinated sheep blood, and the SAP material having a gel-strength that exceeds 18000 Pa.

Furthermore, the inventor has found that a SAP material suitable for use in embodiments of the invention has a degree of cross-linking agent in a range of 3.0 to 7.0 mol %.

That SAP particles having a cross-linking degree in this range works well for absorption of blood is clearly surprising, since it has been thought that a too low expandability is obtained with a cross-linking degree as high as this. However, the inventor has shown that this low expandability is compensated for because the gel-strength results in a high initial absorption capacity for blood, and that this in combination with a suitably chosen wettability on the surface of the SAP material, gives SAP material that is especially suited for absorption of blood or blood-containing fluids.

By choosing a gel-strength and/or a cross-linking degree within this range and a contact angle smaller than 60°, and preferably smaller than 40°, a balance between hydrophilicity on the surface of the SAP and absorption capacity within the SAP is achieved.

Moreover, the SAP used in embodiments of the invention do not only have a high degree of cross-linking, but also have some degree of non-cross-linked, free polymers in their structures. These free polymers do not contribute to the degree of cross-linking, but they improve the gel-strength. Thus, a high osmotic pressure is achieved and, therefore, a high initial absorption capacity. The free polymers give an “imaginary cross-linking”, i.e., they strengthen the osmotic pressure without making the gel harder and less swellable. According to a preferred embodiment, the SAP has a gel-strength above 18000 Pa, more preferably above 25000 Pa, and most preferably above 30000 Pa.

In one embodiment the invention relates to an absorbent product, containing the above-mentioned SAP material, which has an absorption capacity for blood higher than 6 g blood/g SAP, preferably higher than 10 g blood/g SAP, and most preferably higher than 13 g blood/g SAP, with the given absorption capacity values measured at absorption under load (AUL) of 2 kPa. Moreover, it is possible to mix various types of SAP having different absorption capacities in an absorbent product.

In yet another embodiment, the invention relates to an absorbent product, in which the surface of the SAP particles is treated with a surface modifier, such as AlCl₃ and/or Al₂(SO₄)₃. This treatment obtains SAP particles with desired surface properties, which in turn facilitate a good absorption of blood.

Embodiments of the invention also include methods for making and/or using the above-mentioned SAP particles, absorbent body, and absorbent product.

### BRIEF DESCRIPTION OF THE DRAWINGS

FIG. 1 shows an example of a sanitary towel according to an embodiment of the invention.

FIG. 2 shows a graph describing the absorption capacity as a function of the a degree of cross-linking agent for different liquids and SAP materials.

FIG. 3 shows a graph describing the absorption capacity as a function of the SAP gel-strength for different liquids and SAP materials.

### DETAILED DESCRIPTION OF PREFERRED EMBODIMENTS

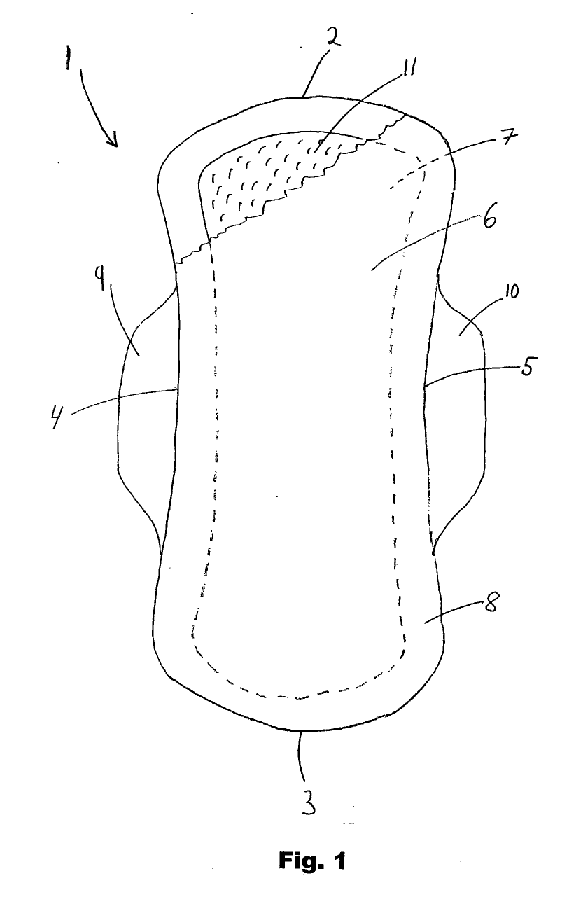

In FIG. 1 a principal sketch of a sanitary towel 1 according to an embodiment of the invention is shown. However, the invention is not limited to sanitary towels, but may also comprise other absorbent products, such as panty liners, incontinence protectors, dressings and tampons, which are intended for absorption of blood or menstrual fluid. In one embodiment, the sanitary towel may include two short sides 2, 3 and two long sides 4, 5. A liquid permeable surface layer 6 is applied on the side of the sanitary towel 1 facing the wearer during use. The liquid permeable surface layer 6 suitably comprises a soft, skin-friendly material. Examples of suitable liquid permeable materials include various types of non-woven fiber materials (non-wovens). Other usable surface layers can include perforated plastic films, nets, knitted, crocheted or woven textiles, and combinations and laminates of the listed material types.

The liquid-blocking backing layer 7 can include a liquid impermeable material. Thin, liquid-proof plastic films are suitable for the purpose, but it is also possible to use materials which initially are liquid permeable, but which are equipped with a coating of plastics, resin, or some other liquid-proof material. Thus, the leakage of liquid from the underside of the absorbent product is prevented. The blocking layer 7 may therefore comprise any material meeting the criteria of liquid impermeability, and having appropriate flexibility and skin-friendliness. Examples of materials that are suitable for use as the blocking layer are plastic films, non-woven and laminates of these. The plastic film can include, for example, polyethylene, polypropylene or polyester. The blocking layer can alternatively include a laminate of a liquid-permeable plastic layer facing the absorbent body, and a non-woven facing the undergarment of the wearer. A construction of that kind provides a leakage secure layer having a textile-like character. The liquid-blocking backing layer 7 may also consist of a vapor-permeable material. A breathable backing layer 7 of that kind may be, for example, a SMS-material (spunbond-meltblown-spunbond) or a breathable plastic film formed of polyethylene. A plastic film of that kind is disclosed in EP-A-283200. In order to keep the breathability also when the material is applied on a product, the underside should not be totally covered with fastening means.

The two wrapping layers (the surface layer and the backing layer 6, 7) may be connected to each other and may optionally form a protruding connecting edge 8 around the contour line of the sanitary towel. The connection may be accomplished with any technique suitable for the purpose, such as gluing, welding or sewing. The two wrapping layers are not necessary, but may in some cases be suitable.

Between the surface layer and the backing layer 6, 7 a thin flexible absorbent core 11 is placed, which may comprise one or more material layers. The absorbent core 11 can be suitably manufactured with one or more layers of cellulose pulp. The pulp may originally be in the form of rolls, bales or sheets, which at the manufacturing of the sanitary towel is dry-defibrated and is transmitted in fluffed form to a pulp mat. The pulp can include super-absorbents, which are polymers having the ability to absorb water or body fluids in an amount of several times their own weight. An alternative to this is to dry-form a pulp mat, such as described in WO94/10956. Examples of other usable absorbent materials are different kinds of natural fibers, such as cotton fibers, peat or the like. Naturally, it is also possible to use absorbent synthetic fibers, or particles of a high-absorbing polymer material of a type which at absorption chemically binds large amounts of liquid during the formation of a liquid-containing gel, or mixtures of natural fibers or synthetic fibers. The absorbent body 11 may further comprise additional components, such as form-stabilizing means, liquid-spreading means, or binders, such as, for example, thermoplastic fibers which have been heat-treated to hold short fibers and particles to a connecting unit. It is also possible to use different types of absorbing foam materials in the absorbent body. A further variant is to manufacture the core only of SAP material.

Preferably, the super-absorbent polymers (SAP) of the invention are manufactured of polyacrylic acid monomers, such as those available from KeboLab, for instance. The monomers are thereafter treated in order to form polymers, as described in further detail in the example below. They are added to the other absorption material in the form of beads, granules, foam, fibers, threads, film or the like. Granules are preferred in the invention, and they are used at an approximate size of 100 to 850 μm. Other manufacturing methods may of course also be used to form the SAP.

Other materials that may be used to produce super-absorbent polymers suitable to use in embodiments of the invention include hydrolyzed starch-acrylonitrile graft copolymers, starch-acrylic acid graft copolymers, saponified vinyl acetate-acrylic ester copolymers, hydrolyzed acrylonitrile copolymers, hydrolyzed acrylamide copolymers, ethylene-maleic anhydride copolymers, isobutylene-maleic anhydride copolymers, poly-(vinylsulfonic acid), poly-(vinylphosphonic acid), poly-(vinylphosphoric acid), poly-(vinylsulfuric acid), sulfonated polystyrene, poly-(aspartic acid), poly-(lactic acid), and mixtures thereof, as well as mixtures of the above mentioned compounds and polyacrylic acid. Naturally, also other material combinations known in the art may be considered, and are thus included in the scope of the invention.

The polymer chains are held together with cross-linkings, which are accomplished by the addition of a cross-linking agent, such as methyl-bisacrylamide (MBA), to the original polymers. Other possible cross-linking agents comprise for example aluminium sulphate, N,N′-methylenebisacrylamide, N,N′-methylenebismethacrylamide, ethylene glycol dimethacrylate, and trimethylolpropane triacrylate. It is to be understood by the skilled person that also other cross-linking agents giving a desired cross-linking are included in the scope of the invention.

According to embodiments of the invention, the cross-linking agent is added to the SAP polymer in a concentration of 3 to 7 mol %, and preferably 4 to 5 mol %. This has been shown to result in especially advantageous absorption properties for blood or menstrual fluid. These values are measured at a load of 2 kPa, using the AUL method (Absorption Under Load) in an AUL cell, which has been modified by exchanging a standard filter with a metal filter (having a mesh size of 100×100 μm). These metal filters have enough large meshes for blood cells to pass (about 10 μm in diameter).

The surface of the SAP particles is treated in a way that it preferably has a certain degree of wettability (hydrophilicity). The wettability is measured by the contact angle. Defibrinated sheep blood is a standard with which the contact angle can be measured. The contact angle (see for example “Lexikon i KEMI”, Gleerups förlag, 1976, 1^{st} edition) specifies the angle between the surface and a water droplet, positioned on the surface. The more hydrophilic the surface, the more compressed the droplet becomes (and the smaller the contact angle becomes). A surface showing a value for the contact angle below 90° is regarded as hydrophilic.

In an embodiment of the invention the contact angle is smaller than 60° as measured with defibrinated sheep blood, with the contact angle preferably being smaller than 40°.

In order for the surface of the SAP particles to exhibit the desired hydrophilicity, the surface is treated with a surface modifier, such as aluminum chloride (AlCl₃) and/or aluminum sulphate (Al₂(SO₄)₃). At treatment of a SAP particle surface with these ionic compounds, the chloride- and/or sulphate ions will dissociate and a binding of the trivalent, positively charged aluminum ion is obtained on the surface. At absorption of water-containing liquids, the water dipole will then be attracted to and bind to the aluminum ion. Other surface modifiers that may be used include silica compounds, such as silica oxides, and organic compounds, such as ethylene carbonate. All surface modifiers giving the desired effect, i.e., a desired contact angle, are fully possible for use in embodiments of the invention.

The SAP particles can be added to the absorbent core in an amount of 1 to 100%, and preferably 10 to 60% of the total weight of the absorbent core, in order to obtain a good spreading and/or absorption capacity.

The absorbent product as illustrated by these embodiments of the invention is especially adapted for absorption of blood or menstrual fluid.

Between the surface layer 6 and the absorbent body 11, an inlet layer may be arranged. The purpose of the inlet layer is to direct liquid into the sanitary towel, and to transport it down to the absorbent body 11. The inlet layer may be a non-woven material of low density.

Fastening means be applied to the underside of the sanitary towel. The fastening means can be glue, but it may also be a mechanical fastener such as hook and loop, push buttons, friction linings, clamping means, or the like. The glue may be applied in one or more strings, or in any other pattern. Alternatively, the whole underside of the sanitary towel 1 can be lined with glue. It is also possible to use a fastening glue that is breathable and to apply it to the entire underside of the product, for it to function as a combined liquid-proof layer and fastening means. Moreover, the fastening means may be glue on a surface facing the body, hydrogels, or nothing at all.

A protection layer may be applied over the fastening means, e.g., when glue is used as the fastening means. The protection layer is preferably a siliconized paper, but also other variants of protection layers are of course possible, such as waxed papers, embossed or release agent-treated plastic film, textile ribbons to fasten to hooks and loops, etc.

The sanitary towel of embodiments of the invention may further also comprise fastening tabs 9 and 10 applied along the long sides of the sanitary towel. The purpose of the tabs is to be folded along the edge of the panty, and thereby keep the sanitary towel in place. Furthermore, other kinds of fastening systems may be used.

Embodiments of the absorbent product of the invention can include a panty liner, a sanitary towel, or an incontinence protector, adapted for absorption of blood or menstrual fluid.

In yet another aspect, the invention relates to the use of an absorbent product described above for the absorption of blood or menstrual fluid.

The examples below shows some preferred embodiments of the SAP material of the invention, and are not be considered as limiting the scope of the invention in any way.

### EXAMPLES

### Example 1

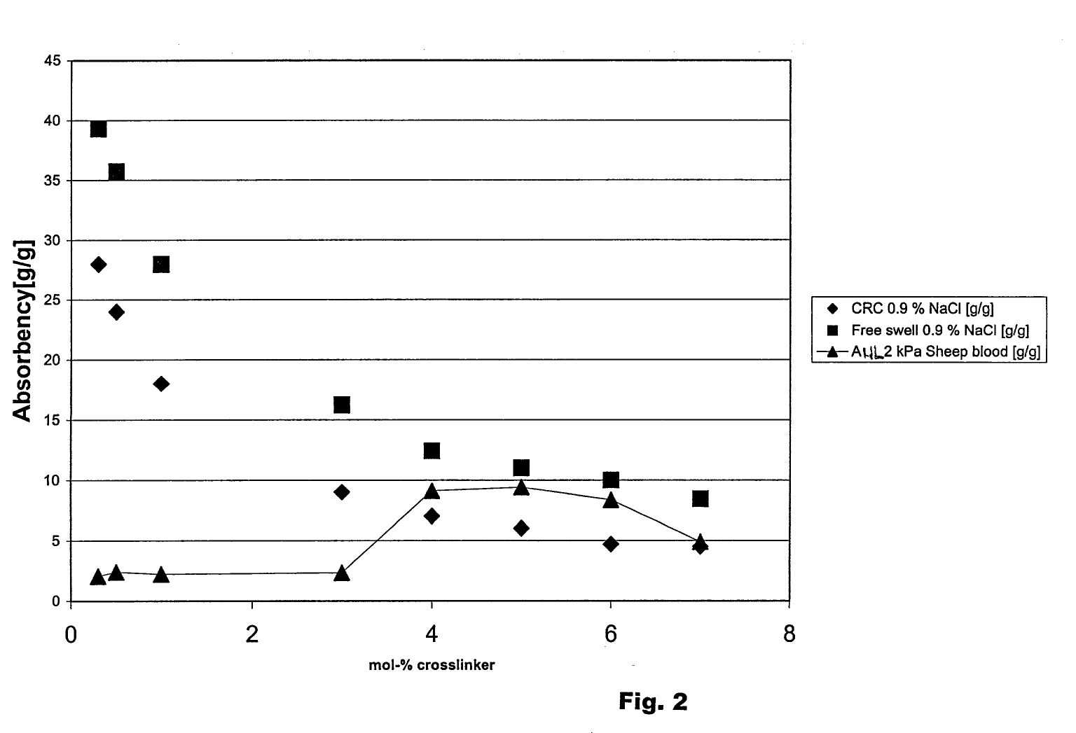

The graph in FIG. 2 shows the relation between absorption capacity (absorbed weight (g)/SAP weight (g)) and the concentration of cross-linking agent (MBA) at different conditions and SAP material. The first curve (diamonds) shows the absorption capacity (according to edana 441.1-99; centrifuge retention capacity) for a CRC-SAP (cellulose) in 0.9% NaCl-solution (i.e., a urine-like solution). The second curve (squares) shows free absorption capacity in 0.9% NaCl solution for a SAP-gel of polyacrylic acid. The absorption capacity is clearly highest at a low degree of cross-linking and decreases rapidly when the cross-linking degree is increased for both SAP-materials in urine-like conditions. The third curve (triangles) shows the absorption capacity for a polyacrylic acid-SAP in sheep blood at AUL 2 kPa. Here a low absorption is obtained at a low cross-linking degree. However, in the interval from 3 to 7 mol % of cross-linking agent, an increased absorption is obtained. At a cross-linking degree of 5 mol % an absorption capacity of almost 10 g/g in defibrinated sheep blood is obtained.

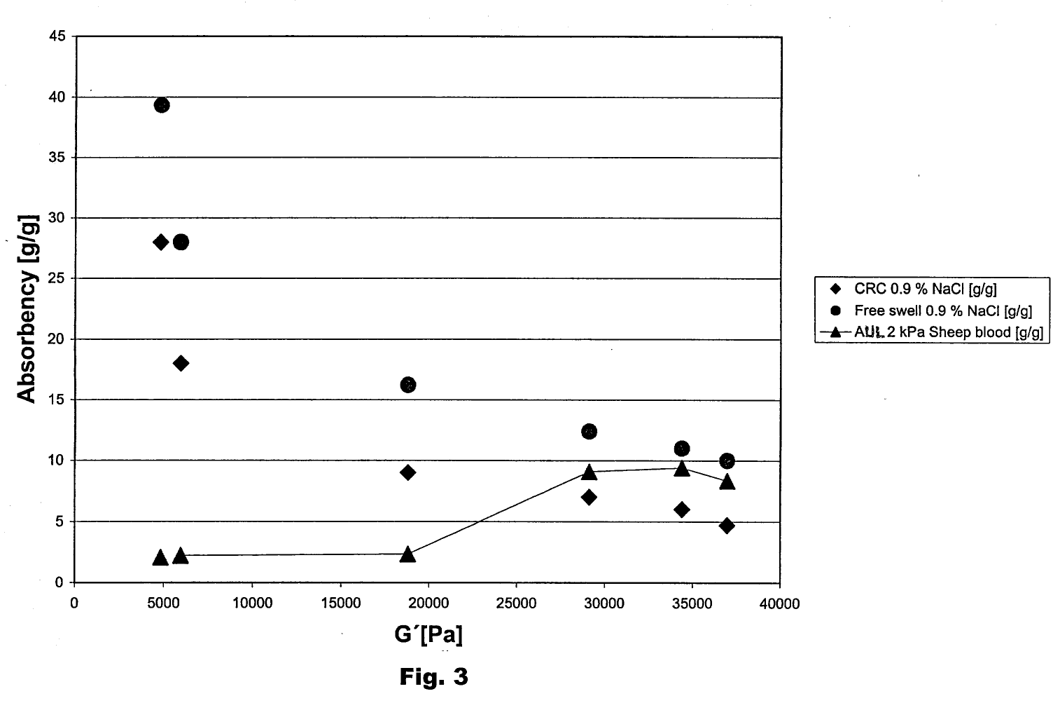

The graph in FIG. 3 shows the relation between absorption capacity and gel-strength (G′ (Pa)). The first curve (diamonds) shows the absorption capacity for a CRC-SAP (cellulose) in 0.9% NaCl-solution (i.e., a urine-like solution). The second curve (circles) shows free absorption capacity in 0.9% NaCl solution for a SAP-gel of polyacrylic acid. The third curve shows the absorption capacity for a polyacrylic acid-SAP in sheep blood at AUL 2 kPa. For curve 1 and 2 the absorption capacity decreases when the gel-strength is increased. For curve 3 a low absorption capacity is obtained with low gel-strength. With a gel-strength above approximately 20000 Pa an increased absorption capacity is achieved, with a top value of between 30000 and 35000 Pa. Here the absorption capacity is almost 10 g/g.

### Example 2

### Manufacture of SAP Particles

To a 25% acrylic acid solution, neutralized to 75%, methylenebisacrylamide is added to a cross-linking degree of 0.1 to 10%. The reaction is initiated with 0.1 mol % of Va-044 (Wako Pure Chemical Industries LTD, Japan) that is neutralized with sodium ions (such as sodium hydroxide or sodium carbonate). The obtained gel is washed, dried, ground (Janke & Kunkel, Analysen Mühce A10) and fractionated to a size of from 100 to 850 mm.

At surface-cross-linking the particles are evaporated in a solution of ethanol, aluminum chloride and sodium hydroxide. This is performed by mixing 25 ml 96% ethanol, 0.1 g water-free AlCl₃ and 300 ml 4M NaOH with 2 g SAP. The mix is allowed to cross-link for about 30 min at 70° C., during stirring. Thereafter, the ethanol is removed under vacuum.

### Example 3

### Material Properties

The gel-strength of the SAP-gel (shear modules) was determined with a TA instruments AR 1000 N. To 0.5 g SAP-sample 2.5 ml 0.9% NaCl solution was added. The elasticity module of the SAP samples were determined with a TA instruments AR 1000 N. The analyses are performed with a 40 mm acrylic parallel plate, cross hatched at 20° C. The procedure is oscillating and the samples were analyzed between 0.1 and 100 Hz. The oscillating shear tension is 10.00 Pa. The measured shear tension is G′ and was measured at 1 Hz.

The contact angle between defibrinated sheep blood and the SAP particles was determined with a DAT 1100 Fibro System AB. The analyses were performed in normal laboratory environment at 22° C. and 50% relative air moisture. For the analyses a particle size of between 100 and 315 μm was chosen.

The resulting contact angles for untreated samples are about 60° with defibrinated sheep blood. After the treatment a contact angle of about 35° is obtained. The AUL values increases from about 10 g/g to about 13 g/g at a decrease of the contact angle from 60° to 35°.

Although only preferred embodiments are specifically illustrated and described herein, it will be appreciated that many modifications and variations of the present invention are possible in light of the above teachings and within the purview of the appended claims without departing from the spirit and intended scope of the invention.

1. An absorbent product comprising:
an absorbent body for absorbing blood,
the absorbent body comprising between about 10% and about 60% by weight of a super-absorbent polymer,
wherein the super-absorbent polymer is cross-linked with between 3.0 and 7.0 mol % of a cross-linking agent,
the super-absorbent polymer having a gel strength of at least 18 kPa,
the super-absorbent polymer having a surface treated with a hydrophilicity surface modifier,
the superabsorbent polymer having a contact angle smaller than 60° as measured with defibrinated sheep blood, and
the absorbent body having an absorption capacity of more than 6 g blood/g super-absorbent polymer under a 2 kPa load.

2. An absorbent product comprising:
an absorbent body for absorbing blood,
the absorbent body comprising a super-absorbent polymer,
wherein the super-absorbent polymer includes between 3.0 and 7.0 mol % of a cross-linking agent,
the super-absorbent polymer having a gel strength of at least 18 kPa,
the superabsorbent polymer having a contact angle smaller than 60° as measured with defibrinated sheep blood, and
the absorbent body having an absorption capacity of more than 6 g blood/g super-absorbent polymer under a 2 kPa load.

3. An absorbent product according to claim 2, wherein the super-absorbent polymer has a surface treated with a hydrophilicity surface modifier.

4. An absorbent product comprising:
an absorbent body for absorbing blood,
the absorbent body comprising at least 1% by weight of a super-absorbent polymer,
wherein the super-absorbent polymer includes between 3.0 and 7.0 mol % of a cross-linking agent,
the super-absorbent polymer having a gel strength of at least 18 kPa, and
wherein the super-absorbent polymer has a surface treated to have a contact angle smaller than 60° as measured with defibrinated sheep blood.

5. An absorbent product comprising:
an absorbent body for absorbing blood,
the absorbent body comprising a cross-linked super-absorbent polymer,
the super-absorbent polymer has a gel strength of at least 18 kPa,
the superabsorbent polymer having a contact angle smaller than 60° as measured with defibrinated sheep blood,
and the absorbent body having an absorption capacity of more than 6 g blood/g super-absorbent polymer under a 2 kPa load.

6. The absorbent product according to claim 5, the super-absorbent polymer being between 10 and 60% of the absorbent body by weight.

7. An absorbent product comprising:
an absorbent body for absorbing blood,
the absorbent body comprising a super-absorbent polymer,
the superabsorbent polymer having a contact angle smaller than 60° as measured with defibrinated sheep blood,
and the super-absorbent polymer having a gel-strength greater than 18 kPa.

8. An absorbent product as in claim 7, further comprising:
a liquid permeable surface layer arranged at one side of the absorbent body, and
a liquid impermeable backing layer arranged at a second side of the absorbent body.

9. The absorbent product according to claim 8, further comprising:
an inlet layer arranged between the surface layer and the absorbent body for transporting liquid to the absorbent body.

10. The absorbent product according to claim 7, wherein the super-absorbent polymer has a gel-strength greater than 25 kPa.

11. The absorbent product according to claim 10, wherein the super-absorbent polymer has a gel-strength greater than 30 kPa.

12. The absorbent product according to claim 7, wherein the contact angle as measured with defibrinated sheep blood is less than 40°.

13. The absorbent product according to claim 7, wherein the contact angle as measured with defibrinated sheep blood is about 35°.

14. The absorbent product according to claim 7, wherein the super-absorbent polymer comprises between 3.0 and 7.0 mol % of a cross-linking agent.

15. The absorbent product according to claim 14, wherein the super-absorbent polymer comprises between 4.0 and 5.0 mol % of a cross-linking agent.

16. The absorbent product according to claim 7, wherein the super-absorbent polymer is cross-linked with between 3.0 and 7.0 mol % of a cross-linking agent.

17. The absorbent product according to claim 16, wherein the super-absorbent polymer is cross-linked with between 4.0 and 5.0 mol % of a cross-linking agent.

18. The absorbent product according to claim 16, wherein the cross-linking agent is methyl-bisacrylamide, aluminum sulphate, N,N′-methylenebisacrylamide, N,N′-methylenebismethacrylamide, ethylene glycol dimethacrylate, or trimethylolpropane triacrylate.

19. The absorbent product according to claim 7, wherein the absorbent body has an absorption capacity of more than 6 g blood/g super-absorbent polymer under a 2 kPa load.

20. The absorbent product according to claim 19, wherein the absorption capacity is at least 10 g blood/g super-absorbent polymer under a 2 kPa load.

21. The absorbent product according to claim 20, wherein the absorption capacity is at least 13 g blood/g super-absorbent polymer under a 2 kPa load.

22. The absorbent product according to claim 7, wherein the absorbent body includes between 1% and 100% by weight of the super-absorbent polymer.

23. The absorbent product according to claim 22, wherein the absorbent body includes between 10% and 60% by weight of the super-absorbent polymer.

24. The absorbent product according to claim 7, wherein the super-absorbent polymer has a surface treated with a hydrophilicity surface modifier.

25. The absorbent product according to claim 7, wherein the super-absorbent polymer has a surface treated with aluminum chloride or aluminum sulphate.

26. The absorbent product according to claim 7, wherein the super-absorbent polymer includes granules with a diameter between 100 and 850 μm.

27. The absorbent product according to claim 7, wherein the super-absorbent polymer includes granules with a diameter between 100 and 315 μm.

28. The absorbent product according to claim 7, wherein the super-absorbent polymer comprises cross-linked polyacrylic acid.

29. The absorbent product according to claim 7, wherein the super-absorbent polymer comprises at least one of a hydrolyzed starch-acrylonitrile graft copolymer, starch-acrylic acid graft copolymer, saponified vinyl acetate-acrylic ester copolymer, hydrolyzed acrylonitrile copolymer, hydrolyzed acrylamide copolymer, ethylene-maleic anhydride copolymer, isobutylene-maleic anhydride copolymer, poly-(vinylsulfonic acid), poly-(vinylphosphonic acid), poly-(vinylphosphoric acid), poly-(vinylsulfuric acid), sulfonated polystyrene, poly-(aspartic acid), or poly-(lactic acid).

30. The absorbent product according to claim 7, wherein the absorbent product is a panty liner, a sanitary towel, an incontinence protector, a dressing, or a tampon for the absorption of blood or menstrual fluid.

31. An absorbent product comprising:
an absorbent body for absorbing blood,
the absorbent body comprising a super-absorbent polymer,
the superabsorbent polymer having a contact angle smaller than 60° as measured with defibrinated sheep blood,
the absorbent body having an absorption capacity of more than 6 g blood/g super-absorbent polymer under a 2 kPa load.

32. An absorbent product as in claim 31, further comprising:
a liquid permeable surface layer arranged at one side of the absorbent body, and
a liquid impermeable backing layer arranged at a second side of the absorbent body.

33. The absorbent product according to claim 32, further comprising:
an inlet layer arranged between the surface layer and the absorbent body for transporting liquid to the absorbent body.

34. The absorbent product according to claim 31, wherein the absorption capacity is at least 10 g blood/g super-absorbent polymer under a 2 kPa load.

35. The absorbent product according to claim 31, wherein the absorption capacity is at least 13 g blood/g super-absorbent polymer under a 2 kPa load.

36. The absorbent product according to claim 31, wherein the super-absorbent polymer has a gel-strength greater than 18 kPa.

37. The absorbent product according to claim 31, wherein the super-absorbent polymer has a gel strength greater than 26 kPa.

38. The absorbent product according to claim 37, wherein the super-absorbent polymer has a gel-strength greater than 30 kPa.

39. The absorbent product according to claim 31, wherein the contact angle as measured with defibrinated sheep blood is less than 60°.

40. The absorbent product according to claim 31, wherein the contact angle as measured with defibrinated sheep blood is less than 40°.

41. The absorbent product according to claim 40, wherein the contact angle as measured with defibrinated sheep blood is about 35°.

42. The absorbent product according to claim 31, wherein the super-absorbent polymer comprises between 3.0 and 7.0 mol % of a cross-linking agent.

43. The absorbent product according to claim 31, wherein the super-absorbent polymer comprises between 4.0 and 5.0 mol % of a cross-linking agent.

44. The absorbent product according to claim 31, wherein the super-absorbent polymer is cross-linked with between 3.0 and 7.0 mol % of a cross-linking agent.

45. The absorbent product according to claim 31, wherein the super-absorbent polymer is cross-linked with between 4.0 and 5.0 mol % of a cross-linking agent.

46. The absorbent product according to claim 44, wherein the cross-linking agent is methyl-bisacrylamide, aluminum sulphate, N,N′-methylenebisacrylamide, N,N′-methylenebismethacrylamide, ethylene glycol dimethacrylate, or trimethylolpropane triacrylate.

47. The absorbent product according to claim 31, wherein the absorbent body includes between 10% and 60% by weight of the super-absorbent polymer.

48. The absorbent product according to claim 31, wherein the super-absorbent polymer has a surface treated with a hydrophilicity surface modifier.

49. The absorbent product according to claim 31, wherein the super-absorbent polymer has a surface treated with aluminum chloride or aluminum sulphate.

50. The absorbent product according to claim 31, wherein the super-absorbent polymer includes granules with a diameter between 100 and 850 μm.

51. The absorbent product according to claim 31, wherein the super-absorbent polymer includes granules with a diameter between 100 and 315 μm.

52. The absorbent product according to claim 31, wherein the super-absorbent polymer comprises cross-linked polyacrylic acid.

53. The absorbent product according to claim 31, wherein the super-absorbent polymer comprises at least one of a hydrolyzed starch-acrylonitrile graft copolymer, starch-acrylic acid graft copolymer, saponified vinyl acetate-acrylic ester copolymer, hydrolyzed acrylonitrile copolymer, hydrolyzed acrylamide copolymer, ethylene-maleic anhydride copolymer, isobutylene-maleic anhydride copolymer, poly-(vinylsulfonic acid), poly-(vinylphosphonic acid), poly-(vinylphosphoric acid), poly-(vinylsulfuric acid), sulfonated polystyrene, poly-(aspartic acid), or poly-(lactic acid).

54. The absorbent product according to claim 31, wherein the absorbent product is a panty liner, a sanitary towel, an incontinence protector, a dressing, or a tampon for the absorption of blood or menstrual fluid.

55. An absorbent product comprising:
an absorbent body for absorbing blood,
the absorbent body comprising a super-absorbent polymer,
the superabsorbent polymer having a contact angle smaller than 60° as measured with defibrinated sheep blood,
the super-absorbent polymer being cross-linked with between 3.0 and 7.0 mol % of a cross-linking agent.

56. The absorbent product according to claim 55, wherein the super-absorbent polymer is cross-linked with between 4.0 and 5.0 mol % of a cross-linking agent.

57. An absorbent product as in claim 55, further comprising:
a liquid permeable surface layer arranged at one side of the absorbent body, and
a liquid impermeable backing layer arranged at a second side of the absorbent body.

58. The absorbent product according to claim 57, further comprising:
an inlet layer arranged between the liquid permeable surface layer and the absorbent body.

59. The absorbent product according to claim 55, wherein the contact angle as measured with defibrinated sheep blood is less than 40°.

60. The absorbent product according to claim 55, wherein the contact angle as measured with defibrinated sheep blood is about 35°.

61. The absorbent product according to claim 55, wherein the super-absorbent polymer has a gel-strength greater than 18 kPa.

62. The absorbent product according to claim 61, wherein the super-absorbent polymer has a gel-strength greater than 25 kPa.

63. The absorbent product according to claim 62, wherein the super-absorbent polymer has a gel-strength greater than 30 kPa.

64. The absorbent product according to claim 55, wherein the cross-linking agent is methyl-bisacrylamide, aluminum sulphate, N,N′-methylenebisacrylamide, N,N′-methylenebismethacrylamide, ethylene glycol dimethacrylate, or trimethylolpropane triacrylate.

65. The absorbent product according to claim 55, wherein the super-absorbent polymer comprises a polyacrylic acid, and the super-absorbent polymer has a gel strength of at least 18 kPa.

66. The absorbent product according to claim 55, wherein the absorbent body has an absorption capacity of more than 6 g blood/g super-absorbent polymer under a 2 kPa load.

67. The absorbent product according to claim 66, wherein the absorption capacity is at least 10 g blood/g super-absorbent polymer under a 2 kPa load.

68. The absorbent product according to claim 67, wherein the absorption capacity is at least 13 g blood/g super-absorbent polymer under a 2 kPa load.

69. The absorbent product according to claim 545 wherein the absorbent body includes between 1% and 100% by weight of the super-absorbent polymer.

70. The absorbent product according to claim 69, wherein the absorbent body includes between 10% and 60% by weight of the super-absorbent polymer.

71. The absorbent product according to claim 55, wherein the super-absorbent polymer has a surface treated with a hydrophilicity surface modifier.

72. The absorbent product according to claim 55, wherein the super-absorbent polymer has a surface treated with aluminum chloride or aluminum sulphate.

73. The absorbent product according to claim 55, wherein the super-absorbent polymer includes granules with a diameter between 100 and 850 μm.

74. The absorbent product according to claim 73, wherein the super-absorbent polymer includes granules with a diameter between 100 and 315 μm.

75. The absorbent product according to claim 55, wherein the super-absorbent polymer comprises a cross-linked polyacrylic acid.

76. The absorbent product according to claim 55, wherein the super-absorbent polymer comprises at least one of a hydrolyzed starch-acrylonitrile graft copolymer, starch-acrylic acid graft copolymer, saponified vinyl acetate-acrylic ester copolymer, hydrolyzed acrylonitrile copolymer, hydrolyzed acrylamide copolymer, ethylene-maleic anhydride copolymer, isobutylene-maleic anhydride copolymer, poly-(vinylsulfonic acid), poly-(vinylphosphonic acid), poly-(vinylphosphoric acid), poly-(vinylsulfuric acid), sulfonated polystyrene, poly-(aspartic acid), or poly-(lactic acid).

77. The absorbent product according to claim 55, wherein the absorbent product is a panty liner, a sanitary towel, an incontinence protector, a dressing, or a tampon for the absorption of blood or menstrual fluid.

78. An absorbent product comprising:
an absorbent body for absorbing blood,
the absorbent body comprising a super-absorbent polymer,
the superabsorbent polymer having a gel-strength greater than 18 kPa,
and the absorbent body having an absorption capacity of more than 6 g blood/g super-absorbent polymer under a 2 kPa load.

79. An absorbent product as in claim 78, further comprising:
a liquid permeable surface layer arranged at one side of the absorbent body, and
a liquid impermeable backing layer arranged at a second side of the absorbent body.

80. The absorbent product according to claim 79, further comprising:
an inlet layer arranged between the surface layer and the absorbent body for transporting liquid to the absorbent body.

81. The absorbent product according to claim 78, wherein the absorption capacity is at least 10 g blood/g super-absorbent polymer under a 2 kPa load.

82. The absorbent product according to claim 78, wherein the absorption capacity is at least 13 g blood/g super-absorbent polymer under a 2 kPa load.

83. The absorbent product according to claim 78, wherein the super-absorbent polymer has a gel-strength greater than 26 kPa.

84. The absorbent product according to claim 83, wherein the super-absorbent polymer has a gel-strength greater than 30 kPa.

85. The absorbent product according to claim 78, wherein the contact angle as measured with defibrinated sheep blood is less than 60°.

86. The absorbent product according to claim 78, wherein the contact angle as measured with defibrinated sheep blood is less than 40°.

87. The absorbent product according to claim 85, wherein the contact angle as measured with defibrinated sheep blood is about 35°.

88. The absorbent product according to claim 77, wherein the super-absorbent polymer comprises between 3.0 and 7.0 mol % of a cross-linking agent.

89. The absorbent product according to claim 78, wherein the super-absorbent polymer comprises between 4.0 and 5.0 mol % of a cross-linking agent.

90. The absorbent product according to claim 78, wherein the super-absorbent polymer is cross-linked with between 3.0 and 7.0 mol % of a cross-linking agent.

91. The absorbent product according to claim 78, wherein the super-absorbent polymer is cross-linked with between 4.0 and 5.0 mol % of a cross-linking agent.

92. The absorbent product according to claim 90, wherein the cross-linking agent is methyl-bisacrylamide, aluminum sulphate, N,N′-methylenebisacrylamide, N,N′-methylenebismethacrylamide, ethylene glycol dimethacrylate, or trimethylolpropane triacrylate.

93. The absorbent product according to claim 78, wherein the absorbent body includes between 1% and 100% by weight of the super-absorbent polymer.

94. The absorbent product according to claim 78, wherein the absorbent body includes between 10% and 60% by weight of the super-absorbent polymer.

95. The absorbent product according to claim 78, wherein the super-absorbent polymer has a surface treated with a hydrophilicity surface modifier.

96. The absorbent product according to claim 78, wherein the super-absorbent polymer has a surface treated with aluminum chloride or aluminum sulphate.

97. The absorbent product according to claim 78, wherein the super-absorbent polymer includes granules with a diameter between 100 and 850 μm.

98. The absorbent product according to claim 78, wherein the super-absorbent polymer includes granules with a diameter between 100 and 315 μm.

99. The absorbent product according to claim 78, wherein the super-absorbent polymer comprises a cross-linked polyacrylic acid.

100. The absorbent product according to claim 78, wherein the super-absorbent polymer comprises at least one of a hydrolyzed starch-acrylonitrile graft copolymer, starch-acrylic acid graft copolymer, saponified vinyl acetate-acrylic ester copolymer, hydrolyzed acrylonitrile copolymer, hydrolyzed acrylamide copolymer, ethylene-maleic anhydride copolymer, isobutylene-maleic anhydride copolymer, poly-(vinylsulfonic acid), poly-(vinylphosphonic acid), poly-(vinylphosphoric acid), poly-(vinylsulfuric acid), sulfonated polystyrene, poly-(aspartic acid), or poly-(lactic acid).

101. The absorbent product according to claim 78, wherein the absorbent product is a panty liner, a sanitary towel, an incontinence protector, a dressing, or a tampon for the absorption of blood or menstrual fluid.

102. An absorbent product comprising:
an absorbent body for absorbing blood,
the absorbent body comprising a super-absorbent polymer,
the super-absorbent polymer being cross-linked with between 3.0 and 7.0 mol % of a cross-linking agent,
and the super-absorbent polymer having a gel-strength greater than 18 kPa.

103. An absorbent product as in claim 102, further comprising:
a liquid permeable surface layer arranged at one side of the absorbent body, and
a liquid impermeable backing layer arranged at a second side of the absorbent body.

104. The absorbent product according to claim 103, further comprising:
an inlet layer arranged between the surface layer and the absorbent body for transporting liquid to the absorbent body.

105. The absorbent product according to claim 102, wherein the super-absorbent polymer has a gel-strength greater than 25 kPa.

106. The absorbent product according to claim 105, wherein the super-absorbent polymer has a gel-strength greater than 30 kPa.

107. The absorbent product according to claim 102, wherein the super-absorbent polymer is cross-linked with between 4.0 and 5.0 mol % of a cross-linking agent.

108. The absorbent product according to claim 102, wherein the cross-linking agent is methyl-bisacrylamide, aluminum sulphate, N,N′-methylenebisacrylamide, N,N′-methylenebismethacrylamide, ethylene glycol dimethacrylate, or trimethylolpropane triacrylate.

109. The absorbent product according to claim 102, wherein the superabsorbent polymer has a contact angle smaller than 60° as measured with defibrinated sheep blood.

110. The absorbent product according to claim 109, wherein the contact angle as measured with defibrinated sheep blood is less than 40°.

111. The absorbent product according to claim 110, wherein the contact angle as measured with defibrinated sheep blood is about 35°.

112. The absorbent product according to claim 102, wherein the absorbent body has an absorption capacity of more than 6 g blood/g super-absorbent polymer under a 2 kPa load.

113. The absorbent product according to claim 102, wherein the absorption capacity is at least 10 g blood/g super-absorbent polymer under a 2 kPa load.

114. The absorbent product according to claim 102, wherein the absorption capacity is at least 13 g blood/g super-absorbent polymer under a 2 kPa load.

115. The absorbent product according to claim 102, wherein the absorbent body comprises between 1% and 100% by weight of the super-absorbent polymer.

116. The absorbent product according to claim 102, wherein the absorbent body includes between 10% and 60% by weight of the super-absorbent polymer.

117. The absorbent product according to claim 102, wherein the super-absorbent polymer has a surface treated with a hydrophilicity surface modifier.

118. The absorbent product according to claim 102, wherein the super-absorbent polymer has a surface treated with aluminum chloride or aluminum sulphate.

119. The absorbent product according to claim 102, wherein the super-absorbent polymer includes granules with a diameter between 100 and 850 μm.

120. The absorbent product according to claim 102, wherein the super-absorbent polymer includes granules with a diameter between 100 and 315 μm.

121. The absorbent product according to claim 102, wherein the super-absorbent polymer comprises cross-linked polyacrylic acid.

122. The absorbent product according to claim 102, wherein the super-absorbent polymer comprises at least one of a hydrolyzed starch-acrylonitrile graft copolymer, starch-acrylic acid graft copolymer, saponified vinyl acetate-acrylic ester copolymer, hydrolyzed acrylonitrile copolymer, hydrolyzed acrylamide copolymer, ethylene-maleic anhydride copolymer, isobutylene-maleic anhydride copolymer, poly-(vinylsulfonic acid), poly-(vinylphosphonic acid), poly-(vinylphosphoric acid), poly-(vinylsulfuric acid), sulfonated polystyrene, poly-(aspartic acid), or poly-(lactic acid).

123. The absorbent product according to claim 10, wherein the absorbent product is a panty liner, a sanitary towel, an incontinence protector, a dressing, or a tampon for the absorption of blood or menstrual fluid.

124. An absorbent product comprising:
an absorbent body for absorbing blood,
the absorbent body comprising a super-absorbent polymer,
the super-absorbent polymer being cross-linked with between 3.0 and 7.0 mol % of a cross-linking agent,
the absorbent body having an absorption capacity of more than 6 g blood/g super-absorbent polymer under a 2 kPa load.

125. The absorbent product according to claim 124, wherein the absorption capacity is at least 10 g blood/g super-absorbent polymer under a 2 kPa load.

126. The absorbent product according to claim 124, wherein the absorption capacity is at least 13 g blood/g super-absorbent polymer under a 2 kPa load.

127. The absorbent product according to claim 124, wherein the super-absorbent polymer is cross-linked with between 4.0 and 5.0 mol % of a cross-linking agent.

128. The absorbent product according to claim 124, wherein the cross-linking agent is methyl-bisacrylamide, aluminum sulphate, N,N′-methylenebisacrylamide, N,N′-methylenebismethacrylamide, ethylene glycol dimethacrylate, or trimethylolpropane triacrylate.

129. The absorbent product according to claim 124, the super-absorbent polymer having a gel-strength greater than 18 kPa.

130. The absorbent product according to claim 124, wherein the super-absorbent polymer has a gel-strength greater than 25 kPa.

131. The absorbent product according to claim 124, wherein the super-absorbent polymer has a gel-strength greater than 30 kPa.

132. The absorbent product according to claim 124, wherein the super-absorbent polymer has a contact angle smaller than 60° as measured with defibrinated sheep blood,

133. The absorbent product according to claim 132, wherein the contact angle as measured with defibrinated sheep blood is less than 40°.

134. The absorbent product according to claim 133, wherein the contact angle as measured with defibrinated sheep blood is about 35°.

135. An absorbent product as in claim 124, further comprising:
a liquid permeable surface layer arranged at one side of the absorbent body, and
a liquid impermeable backing layer arranged at a second side of the absorbent body.

136. The absorbent product according to claim 135, further comprising:
an inlet layer arranged between the surface layer and the absorbent body for transporting liquid to the absorbent body.

137. The absorbent product according to claim 124, wherein the absorbent body comprises between 1% and 100% by weight of the super-absorbent polymer.

138. The absorbent product according to claim 124, wherein the absorbent body includes between 10% and 60% by weight of the super-absorbent polymer.

139. The absorbent product according to claim 124, wherein the super-absorbent polymer has a surface treated with a hydrophilicity surface modifier.

140. The absorbent product according to claim 124, wherein the super-absorbent polymer has a surface treated with aluminum chloride or aluminum sulphate.

141. The absorbent product according to claim 124, wherein the super-absorbent polymer includes granules with a diameter between 100 and 850 μm.

142. The absorbent product according to claim 124, wherein the super-absorbent polymer includes granules with a diameter between 100 and 315 μm.

143. The absorbent product according to claim 124, wherein the super-absorbent polymer comprises an at least partially cross-linked polyacrylic acid.

144. The absorbent product according to claim 124, wherein the super-absorbent polymer comprises at least one of a hydrolyzed starch-acrylonitrile graft copolymer, starch-acrylic acid graft copolymer, saponified vinyl acetate-acrylic ester copolymer, hydrolyzed acrylonitrile copolymer, hydrolyzed acrylamide copolymer, ethylene-maleic anhydride copolymer, isobutylene-maleic anhydride copolymer, poly-(vinylsulfonic acid), poly-(vinylphosphonic acid), poly-(vinylphosphoric acid), poly-(vinylsulfuric acid), sulfonated polystyrene, poly-(aspartic acid), or poly-(lactic acid).

145. The absorbent product according to claim 124, wherein the absorbent product is a panty liner, a sanitary towel, an incontinence protector, a dressing, or a tampon for the absorption of blood or menstrual fluid.
